(19) United States
(12) Patent Application Publication
(10) Pub. No.: US 2016/0045717 A1
(43) Pub. Date: Feb. 18, 2016

(54) CATHETER BALLOON PROVIDED WITH MICROBOREHOLES AND A METAL MESH

(71) Applicant: IPPYRAMIDS GMBH, Ennetbürgen (CH)

(72) Inventor: Eberhard Issendorff, Rethen (DE)

(21) Appl. No.: 14/784,001

(22) PCT Filed: Apr. 10, 2014
(86) PCT No.: PCT/EP2014/057255

§ 371 Date: Oct. 12, 2015

(30) Foreign Application Priority Data
Apr. 13, 2013 (EP) 13163646.6

(51) Int. Cl. A61M 25/10; A61L 29/16; A61L 29/02

(52) U.S. Cl. CPC A61M 25/1002; A61L 29/02; A61L 29/16; A61L 2400/16; A61L 2300/416; A61M 2025/105; A61M 2025/1086

(57) ABSTRACT

The present invention relates to a balloon catheter having a catheter balloon with micro drillings and a metal mesh and particularly to balloon catheters for the cardiac field for minimizing or preventing restenosis. Active agent solutions may be administered through the micro drillings such as using an injection catheter.

The present invention relates to balloon catheters having a catheter balloon with micro drillings and a metal mesh and particularly to balloon catheters for the cardiac field for minimizing or preventing restenosis. Active agent solutions may be administered through the micro drillings such as using an injection catheter, which preferably can be used simultaneously for the dilatation of the catheter balloon.

In the prior art vascular supports, such as stents, are particularly used for reducing or preventing of restenosis in a dilated vessel, in particular in a blood vessel.

Until today, it is not known what the radial strength of the stents has to be to keep open a vessel with a dilated stenosis.

Presumably, only very small forces are required for this purpose, because no (large) forces act from the outside on the coronary vessels and anyway inside a positive pressure (blood pressure) exists. In many cases, the mere (elevated) blood pressure causes aneurysms or dissections of the vessel wall.

If, as is often the case, unilateral stenosis with hard calcifications (no soft deposits) exists, the vessel is not equally, radially opened with the pure balloon dilation, but stretched asymmetrical and often overstretched. At worst, the calcified vessel side is not stretched and the opposite side of the vessel is overstretched or even ruptured, i.e. tearing the vessel wall or dissection.

The overstretched (scribed) or damaged vessel wall is prone to increased proliferation and thus to frequent re-closure, which is referred to as restenosis. The pure balloon dilatation (without stent) of coronary vessels, therefore, leads to an increased restenosis rate of approximately 30%.

For this reason, a balloon dilatation is preferably carried out with the stent, i.e. a stent is mounted on the catheter balloon, i.e. crimped, which is expanded by the balloon dilatation and remains in the vessel during deflation of the catheter balloon.

For further reduction of the restenosis rate, anti-inflammatory, cytostatic, cytotoxic, anti-proliferative, anti-microtubule, anti-angiogenic, anti-neoplastic, anti-migrative, athrombogeneous or anti-thrombogenic agents as anti-restenosis agent are often administered via the catheter balloon. For this, the catheter balloons are coated with either the drug or a drug-containing coating or a drug solution is released through the catheter balloon or cavities in the catheter balloon.

Coated balloon catheter with coated catheter balloons and injection catheter are known in the art. WO 2010/024871 A1 discloses for example a needle injection catheter for administration of therapeutic agents from deposits formed in the balloon sleeve. The European patent EP 2269664 B1 protects catheter balloons coated with a citrate ester and paclitaxel.

According to some clinical trials, the pure balloon dilatation can be significantly improved if simultaneously a medicament such as paclitaxel is released, which prevents proliferation of the vessel or favors the healing process.

Currently, there are several approved balloon catheters which are externally coated with a drug (coated balloon catheter). For the short time during which the balloon is fully dilated (30-120 seconds or shorter and twice opening) a certain dose of an active agent should diffuse into the vessel wall and act there for several days/weeks.

The problem is the precise measurement and control of drug delivery (dosage). As early as when advancing the balloon catheter through the vessels to the stenosis parts of the coating are lost by diffusion or even delamination. Only the active agents that are in the balloon folds are reliably transported to the stenosis. How much active agent actually diffuses into the vessel wall, also depends on the duration of the dilation and the characteristic/capacity of the occluded vascular segment (calcification etc.).

One variant of balloon catheters for a better dosed drug delivery are balloon catheter, where an inner balloon is surrounded by an outer. In the space between there is a solution of a drug, which is pressed out of the outer balloon containing relatively large holes during dilation of the inner balloon.

In addition, balloon catheters are known, which are covered with a metal mesh. The company TriReme Medical Inc. distributes a balloon catheter branded Chocolate® having a catheter balloon which is coated with a metal grid.

In addition, WO 9955285 A2 discloses inter alia, a so-called scoring balloon with a network as a coating on the catheter balloon. The reticulated grid can on one hand be used for releasing an active agent and on the other hand should be adapted to produce microcracks in calcified stenosis, which should reduce vascular lesions and also recoil of the vessel.

The prior art further distinguishes three types of dilatation balloons, namely the so-called “compliant”-balloons, “semi-compliant-balloons and “non-compliant”-balloons.

“Non-compliant” balloons are made of a solid (rigid) material that expands not or only very slightly, even using high pressure. They have a predetermined final diameter and are used when treating highly calcified stenosis that requires high power, respectively a high pressure, for dilatation. If the stenosis cracks and the vessel is opened over-dilatation of the vessel can be avoided.

“Semi-compliant” balloons have a slight stretchability at a certain pressure (for example at a pressure within the range of 6 MPa to 10 MPa). The cardiologist can thus adapt the balloon at the vessel diameter by choice of the pressure. But when treating stiff stenosis, there is no guarantee that the balloon dilates uniformly over the length of the occluded vessel to the final diameter.

“Compliant” balloons are balloons, which dilates nearly arbitrarily depending on the pressure. Such balloons are in general not used for the vessel dilation, since their dilatation over the length of the balloon is not controllable. Possibly, they expand only upstream and downstream of a stenosis and in addition overstretch the vessel there.

It is the objective of the present invention to provide a balloon catheter for active agent application which uniformly opens in particular calcified stenosis and thereby prevents the over-dilatation of the non-calcified regions of the vessel and is additionally able to release a defined amount of an anti-restenotic agent.

This objective is solved by the technical teaching of the independent claims of the present invention. Further advantageous embodiments of the invention result from the dependent claims, the description, the figures and the examples.

Thus, the present invention relates to a balloon catheter with a catheter balloon, wherein the catheter balloon has micro drillings and is covered with a metal mesh and the catheter balloon is formed for receiving an active agent solution, which can be administered through the micro drillings.

With other words, the present invention refers to a balloon catheter with a catheter balloon, wherein the catheter balloon has micro drillings and the catheter balloon is covered with a metal mesh and the catheter balloon can be filled with an active agent solution which can be applied through the micro drillings.

A balloon catheter with a catheter balloon, wherein the catheter balloon has micro drillings and the catheter balloon is covered with a metal mesh and the catheter balloon can be filled with an active agent solution and can be dilated and a predetermined volume of the active agent solution is releasable through the micro drillings during the dilatation is a subject matter of the present invention.

A balloon catheter is preferably a dilatation catheter, PTCA-catheter (Percutaneous Transluminal Coronary Angioplasty Catheter), a PTA-catheter, an infusion catheter, an injection catheter or an angioplasty catheter in particular for blood vessels and further preferred for coronary blood vessels.

According to the invention the catheter balloon is further a “semi-compliant” or “non-compliant” catheter balloon, but also, contrary to the actual clinical usage, a “compliant” catheter balloon.

A catheter balloon is characterized in that the balloon in the deflated or compressed state is preferably folded and has initially a diameter as small as possible (see FIG. 1). During dilation, the pressure in the interior of the catheter balloon increases and the catheter balloon increases its diameter by unfolding.

The balloon sleeve of a “semi-compliant” catheter balloon does not initially dilate. Only after complete unfolding and after achievement of a diameter (the so-called nominal pressure) that is present at a pressure being sufficient to fully deploy the catheter balloon, a limited, additional expansion of the catheter balloon takes place with further pressure increase, because the balloon sleeve is able to stretch within defined limits.

A “semi-compliant” PTCA catheter balloon thus increases its folded initial diameter of preferably 0.7-0.9 mm at nominal pressure by approximately 2.5 times to approximately 6.0 times to approximately 1.5 to 5 mm because of the deployment.

After completion of deployment, a further enlargement of the diameter by typically 10%-20% (based on the diameter in the fully inflated condition) occurs at pressures above the nominal pressure due to stretching of the balloon sleeve.

Thereby diameter refers to the outer diameter, thus from one surface of the catheter balloon to the opposite surface of the catheter balloon in an idealized round shape so as shown by FIG. 1.

Thus, a certain pressure is required in order to fully deploy the catheter balloon. The minimum pressure required to achieve a complete unfolding is also referred to as the nominal pressure. A further increase of the diameter over the diameter present with complete unfolding is achieved by increasing the pressure over the nominal pressure.

In general, pressures of 5 MPa-8 MPa are necessary to produce a complete unfolding of “semi-compliant” PTCA catheter balloons. Over a pressure of 5-8 MPa the balloon sleeve begins to further stretch such as a balloon and the catheter balloon enlarges its diameter further within a defined limit, because it is a “semi-compliant” catheter balloon and not a “compliant” catheter balloon which would further enlarges its diameter at increasing pressure until it bursts.

In the prior art a “compliant” PTCA catheter balloon is usually not used for vasodilatation because his balloon sleeve is arbitrary and uncontrollable extensible, typically up to 100% as well as up to 500% of the nominal diameter. It consists of a rubber-like, highly stretchable material and has only a very small nominal pressure up to 2 bar.

A “compliant” PTCA catheter balloon increases its folded initial diameter of preferably 0.7-0.9 mm at a nominal pressure to approximately 2.5 times to approximately 6.0 times to approximately 1.5 to 5 mm due to the deployment. By increasing the pressure, however, a further increase in the diameter can take place out by stretching the balloon sleeve up to the burst pressure of the catheter balloon, therefore, “compliant” PTCA catheter balloons are not used in practice. According to the invention their use is, however, possible because the metal mesh allows only a limited maximum elongation, predetermined by the mesh structure. Thereby the metal mesh must be so close meshed that the balloon sleeve cannot be pushed out cushion-shaped between the struts of the mesh and is present in front of the struts. The balloon pressure can be significantly increased up to the amounts of the “semi-compliant”balloons. In this context: the closer the metal mesh, the higher the selectable balloon pressure.

At nominal pressure the “compliant” PTCA balloon catheter is, thus, fully deployed and the surrounding metal mesh has led to a uniform deployment, so that also calcified stenosis are dilated without overstretching the non calcified vessel segment. The surrounding metal mesh is completely or almost completely expanded at the nominal pressure and an additional pressure increase within the catheter balloon results in a slight elongation of the balloon sleeve, nevertheless, which is restricted by the maximum expanded metal mesh, wherein said metal mesh is so close meshed that the balloon sleeve cannot be pressed through the meshes. Thus, a further increase in pressure no longer results in a further, theoretically possible, elongation of the balloon sleeve being prevented by the metal mesh, but this further increase in pressure will be used specifically for the administration of an active agent solution by the micro drillings there through, as explained further below.

Provided, according to the invention, a “non-compliant” PTCA catheter balloon is used, then an elongation of the sleeve does not take place or takes place only minimally. The catheter balloon is inflated under pressure and unfolds during that process until it reaches its maximum diameter at the nominal pressure. At nominal pressure, the metal mesh is preferably fully expanded, too and has caused a uniform dilatation of a calcified stenosis. The balloon catheters according to the invention are particularly suitable for dilatation of calcified and highly calcified stenosis because they allow a uniform dilatation of the calcified and obstructed vessel segment without overstretching the non calcified vascular region. In regard to a “non-compliant” PTCA catheter balloon an additional pressure increase beyond the nominal pressure does not result in further increase in diameter, even not in a slight increase, but only in an increase in pressure in the interior of the catheter balloon. But this further increase in pressure can specifically be used for the administration of an active agent solution through the micro drillings, as explained further below.

According to the invention, the catheter balloon is provided with micro drillings, wherein the micro drillings comprise continuous drillings or micro blind holes. The continuous drillings are in fact micro openings in the balloon sleeve, whereas the micro blind holes are micro drillings, which do not completely penetrate the balloon sleeve. Therefore, the continuous drillings penetrate the balloon sleeve and allow the flow of an active agent solution even at lower pressures, whereas the micro blind holes are openings in the balloon sleeve, whose bottom is directed to the balloon inner lumen and is closed by a thin membrane that bursts only when achieving a defined pressure whereby continuous micro drillings are created which allow the release of active agent solution.

Thus, according to one aspect of the present invention, the catheter balloon is provided with continuous micro drillings, which have a diameter so that no or only a small amount of liquid can leak from these continuous micro drillings until a minimal pressure is reached, which preferably corresponds to the nominal pressure. This means that from the interior of the catheter balloon no or no appreciable amount of liquid or solution can flow through the continuous micro drillings and only with a certain pressure the continuous micro drillings are passable for the liquid or solution. This important aspect of the invention is described in more detail below.

According to the invention the balloon sleeve of the catheter balloon is provided with micro drillings, which may preferably pass perpendicular to the longitudinal axis of the catheter balloon through the balloon sleeve and allow a fluid to release from the interior of the catheter balloon through said micro drillings to the outside. The passage of an active agent solution from the balloon interior through the micro drillings to the outside can theoretically take place at any time in regard to the continuous micro drillings and in regard to the micro blind holes only after rupture of the membrane at the bottom of the micro blind holes.

The micro drillings are defined drillings, which were generated by means of laser, and preferably have a round shape. However, the micro drillings are no channels in a porous balloon material and also no other non-rectilinear channels in the balloon sleeve. In addition, the individual micro drillings are not connected to each other, so that a three-dimensional network of channels results. It is crucial that the micro drillings are defined and reproducible forms, which were subsequently incorporated into the material of the balloon sleeve and that they are no structures that are formed during the preparation of the balloon sleeve, such as porous structures or flaws.

For the generation of the micro drillings preferably an ultra-short pulse laser (USP) laser is used, and alternatively, an excimer laser is used. Only using these newer kinds of lasers, it is possible to drill such small holes in the balloon sleeve. These lasers have extremely short pulse durations (nano, pico, femto seconds), very high pulse peak power and a short wavelength of the laser radiation, typically in the UV range. Only the combination of these characteristics of the laser beams allows drillings in extremely thin polymers having diameters such as the catheter balloon sleeves of a few microns without significant thermal negative influences to the marginal zones of the drillings or to the balloon sleeves per se.

Alternatively, the drillings can also be made by ion beam drilling (s. U.S. Pat. No. 6,585,926 B1 and U.S. Pat. No. 7,115,299 B2). But when using ion beam drilling the positioning of the micro drillings is only badly controllable.

It has been shown in prior art using balloons without metal mesh that depending on the type of the balloon problems exist in regard to the diameter of the micro drillings and the underlying drug delivery.

In regard to a non-compliant balloon almost no material expansion of the balloon sleeve is possible. Consequently, the micro drillings have to be so large that liquid can flow. For a given drilling size the higher the pressure is the more fluid flows through the micro drillings. Here, it is necessary to select a diameter of the micro drillings, which ensures that up to the nominal pressure no or only minimum of liquid flows through the micro drillings during the dilatation of the catheter balloon, and only at a pressure equal to or greater than the nominal pressure, the micro drillings allow to release the liquid of the interior of the catheter balloon. The continuous micro drillings of the invention are therefore designed in a way that no or almost no liquid, such as a active agent-containing contrast medium solution, pass them at a pressure below the nominal pressure, which means, the continuous micro drillings are nearly closed and become only passable for a liquid at a pressure in the range of the nominal pressure. The amount of active agent or the volume of the active agent-containing solution which is to be applied can be controlled by the pressure in the range of the nominal pressure and by the duration of the application. If the nominal pressure is, for example, 10 MPa, so at this pressure a defined amount of an active agent is applied in a given volume of contrast agent solution during dilation within a certain time interval (30 seconds for example). If more active agent should be applied, then the pressure can be increased beyond the nominal pressure e.g. to 11 MPa, so that within the same time interval more contrast agent solution flows through the micro drillings. Upon further increase in pressure, the volume of applied active agent-containing contrast agent solution increases continuously, so that by this pressure control in conjunction with the time of dilatation and the concentration of the active agent in the contrast medium a precisely defined amount of the active agent can be administered. In this manner, this is not possible with any PTCA balloon catheter of the prior art.

Using a compliant balloon (according to the prior art without metal mesh), it is generally very difficult to achieve a uniform dilatation of the balloon, so that “compliant”-balloons have not been for dilatation catheter so far.

In regard to the “semi-compliant” and “compliant” balloon choosing the bore size of the micro drillings is important because the micro drillings dilate under pressure. If the micro drillings are too small, a high pressure must be established (so that active agent is delivered), with which the vessel is optionally overstretched. If the micro drillings are too large, too much active ingredient is released at the required high balloon pressure. The right size, that is, to find the correct diameter for the micro drillings is therefore essential to the invention. Because the drillings in the compliant and semi-compliant balloons dilate under pressure, the drilling diameter should be chosen smaller than in regard to the non-compliant balloons whose drillings do not dilate under pressure.

This problem known in the art for injection balloons is prevented by the mesh provided according to the invention. The metal mesh can be expanded up to a certain outer diameter and envelops the dilatable catheter balloon so close that an additional increase in pressure can not lead to a further expansion of the balloon sleeve. Thus, an overextension of the vessel or an uneven dilatation of a calcified vessel part can be avoided and a further increase in pressure on the other hand increases the volume of the active agent solution applied through the micro drillings.

According to the invention the individual micro drillings and preferably the continuous micro drillings have an inside diameter of 0.5 μm-5.0 μm, preferably 0.6 μm-4.0 μm, more preferably from 0.7 μm-3.6 μm, further preferably from 0.8 μm-3.3 μm, still more preferably from 0.9 μm-3.0 μm, still more preferably from 1.0 μm-2.5 μm, still more preferably from 1.1 μm-2.4 μm, even more preferably from 1.2 μm-2.3 μm, still more preferably from 1.3 μm-2.2 μm, still more preferably from 1.4 μm-2.1 μm and most preferably from 1.5 μm-2.0 μm. The inner diameter refers to an idealized round shape of the micro drillings. If the inner diameter of the micro drilling is not constant over the depth the inner diameter refers to the smallest inner diameter.

Therefore, the following features of the continuous micro drillings are essential to the invention:
a) The micro drillings are selected so small that a significant release of the active agent only takes place at a high pressure.
b) An overextension of the vessel (through a catheter balloon being too much dilated by pressure) is prevented by the metal mesh, for example a NiTi supporting net which allows only a maximum predetermined size, i.e. a maximum diameter of the dilated catheter balloon.
c) During pressure reduction in the course of the deflation of the catheter balloon and in particular of a semi-compliant or compliant catheter balloon, the micro drillings are shrink to the original diameter and do not allow any additional release of the active agent-containing solution or allow only a minor release.

The present invention thus also relates to a balloon catheter having a catheter balloon, wherein the catheter balloon has micro drillings and the catheter balloon is covered with a metal mesh and the catheter balloon can be filed with an active agent solution and/or can be dilated and the active agent solution can be applied through the micro drillings and the micro drillings have a diameter of 0.5 μm up to 20 μm and the catheter balloon has 0.2 to 10 micro drillings per mm².

The micro drillings are preferably only in the cylindrical part of the catheter balloon and not at both ends. Preferably, the micro drillings are distributed nearly uniformly in the central part of the catheter balloon. The central part is defined as 80% of the total length of the catheter balloon defined and 10% of the total length at the beginning and 10% of the total length at the end of the catheter balloon are defined as the edge regions which have significantly less (namely only 50% to 1% of the number of micro drillings in the cylindrical central part) or even no micro drillings

Furthermore, the present invention relates to a balloon catheter having a catheter balloon, wherein the catheter balloon has micro drillings and the catheter balloon is covered with a metal mesh and the catheter balloon can be filled with an active agent solution and/or can be dilated and the active agent solution can be applied through the micro drillings, wherein the micro drillings are continuous micro drillings and the micro drillings have a diameter of 0.5 μm up to 10.0 μm, preferably 0.5 μm to 5.0 μm and more preferably from 1.0 μm to 5.0 μm and the catheter balloon has 1 to 10 continuous micro drillings per mm².

The term “continuous” as used herein means that the micro drillings pass through the balloon sleeve of the catheter balloon and thus the active agent solution contained in the interior of the catheter balloon can leak through these micro drillings from inside to outside.

This is different in regard to the micro blind holes. These are micro drillings, which are not continuous, so do not completely penetrate the balloon sleeve of the catheter balloon, but at the bottom of the micro blind hole a thin layer of material or a thin membrane remains, so that the micro blind hole is a non-continuous micro drilling and during dilatation an active agent solution contained inside in the catheter balloons can leak only when this thin layer of material or this thin membrane ruptures during dilatation due to the established pressure.

According to the invention, thus, the catheter balloon can be provided with micro blind holes instead of continuous micro drillings, wherein the balloon sleeve is in this case not completely perforated (in contrast to the continuous micro drillings). Herein a micro blind hole refers to a drilling, which does not completely perforate the balloon sleeve, so it has only a certain depth. The depth of the blind hole should preferably be at least 70% of the balloon wall thickness, more preferably at least 80% and even more preferably at least 90% of the balloon wall thickness.

The individual micro blind holes preferably have an inner diameter of 1.0 μm-30.0 μm, preferably 1.5 μm-25.0 μm, more preferably from 2.0 μm-20.0 μm, still more preferably of 2.3 μm-15.0 μm, still more preferably from 2.5 μm-10.0 μm, still more preferably from 2.7 μm-8.0 μm, still more preferably from 2.9 μm-6.0 μm, and most preferably from 3.0 μm-4.0 μm. The inner diameter refers to an idealized round shape of the micro blind holes. If the inner diameter along the depth of the micro hole should not be constant the inner diameter refers to the smallest internal diameter.

Preferably, the micro blind holes are produced by ablating using laser radiation in particular by means of ultra-short-pulse lasers (USP lasers) and alternatively by means of excimer laser. Alternatively, the blind holes can also be produced by ion beam drilling.

The remaining balloon wall thickness at the bottom of the micro blind holes is like a small membrane. Upon pressure admission to the catheter balloon with nominal pressure or with a short pressure peak, the wafer-thin remaining hole wall strength (membranes), which separate the micro-blind hole from the internal volume of the catheter balloon, burst and, thus, open the drillings and release the active agent.

That means by bursting or by rupture of the remaining material layer at the bottom of a micro-blind hole a continuous micro drilling is formed from the micro blind hole.

The micro blind holes have, compared to the continuous micro drillings three advantages:
a) As long as the remaining balloon wall strength in the micro blind holes does not burst, it is reliably that no active agent is released.
b) The catheter balloon can be subjected to a higher pressure, before any drug is released.
c) When using micro blind holes larger cross-sections of the drillings can be drilled. Despite the large cross-sections of the micro blind hole, a high balloon pressure can be used, i.e. the balloon can apply a high force to open the occluded vessel.

The present invention, therefore, also relates to a balloon catheter having a catheter balloon, wherein the catheter balloon has micro drillings and the catheter balloon is covered with a metal mesh and the catheter balloon can be filled with an active agent solution and/or can be dilated, wherein the micro drillings are micro blind holes which extend in the balloon sleeve up to a depth of at least 70%, preferably at least 80%, more preferably at least 90% of the wall thickness of the balloon sleeve of the catheter balloon.

The present invention relates further to a balloon catheter having a catheter balloon, wherein the catheter balloon has micro drillings and the catheter balloon is covered with a metal mesh and the catheter balloon can be filled with an active agent solution and/or can be dilated, wherein the micro drillings are micro blind holes which extend in the balloon sleeve up to a depth of at least 70%, preferably at least 80%, more preferably at least 90% of the wall thickness of the balloon sleeve of the catheter balloon and balloon sleeve located between the micro blind hole and the inside of the catheter balloon bursts at a pressure of 8 MPa, preferably at 6 MPa, further preferably at 5 MPa.

Herein, the balloon sleeve located between the micro blind hole and the inside of the catheter balloon is also referred to as a thin layer of material or thin membrane and is the remaining material of the balloon sleeve at the bottom of each micro blind hole. This is the material which has been removed in a continuous micro drilling in comparison to a micro blind hole. This material layer remained at the bottom of a micro blind hole function just like a membrane and is a so-called predetermined breaking point, i.e. this material layer ruptures at a certain pressure developed during the dilation of the catheter balloon.

Thus, the present invention also relates to a balloon catheter having a catheter balloon, wherein the catheter balloon has micro drillings and the catheter balloon is covered with a metal mesh and the catheter balloon can be filled with an active agent solution and/or can be dilated, wherein the micro drillings are micro blind holes which extend in the balloon sleeve up to a depth of at least 70%, preferably at least 80%, more preferably at least 90% of the wall thickness of the balloon sleeve of the catheter balloon and can burst. In addition, the active agent solution can be applied there trough after bursting of the micro blind holes being able to burst.

Thus, the present invention also relates to a balloon catheter having a catheter balloon, wherein the catheter balloon has micro drillings and the catheter balloon is covered with a metal mesh and the catheter balloon can be filled with an active agent solution and/or can be dilated, wherein the micro drillings are micro blind holes which extend in the balloon sleeve up to a depth of at least 70%, preferably at least 80%, more preferably at least 90% of the wall thickness of the balloon sleeve of the catheter balloon and have a diameter of 2.0 μm to 20.0 μm and preferably the catheter balloon has 0.2 to 6 micro blind holes per mm².

The catheter balloons with micro blind holes are preferably semi-compliant and particularly preferred non-compliant balloons. The material of the non-compliant balloons is not extensible and high balloon pressures can made them burst particularly safe.

The micro blind holes may have the same drilling diameter as the continuous micro drillings but, preferably, they have something larger drilling cross sections, since the remaining bore wall thickness (membrane) having a larger cross-section may be easier to burst. Larger cross-sections of the drillings can also be realized, because the membrane of the micro blind holes, even for large hole diameters, allows a high pressure to be set up in the catheter balloon respectively prevents undesired release of the active agent even at low pressures.

The individual micro blind holes are slightly larger than the continuous drillings and according to the invention they have an inner diameter of 0.1 μm-10.0 μm, preferably from 1 μm-6.0 μm, more preferably from 2 μm-5 μm. The inner diameter refers to an idealized round shape of the micro blind holes. Provided that the inner diameter should not be constant over the depth of the blind hole the inner diameter refers to the smallest internal diameter.

Therefore, it is essential to the invention that the micro blind holes have the following features:
a) The drilling diameter or cross-section of the micro blind holes is so large that a burst of the remaining balloon wall thickness (membrane) occurs for sure at an increased pressure (nominal pressure) or at a pressure peak.
b) An overextension of the vessel (by a catheter balloon being over dilated using pressure) is prevented by the metal mesh, for example, a NiTi supporting net, which allows only a maximum predetermined size, i.e. a maximum diameter of the dilated catheter balloon.

The number of micro drillings per mm² surface area of the balloon is preferably between 0.2 and 10, more preferably between 0.5 and 9, or between 1 and 8. In the balloon catheter, the number of continuous micro drillings per mm² surface is between 0.5 and 10, preferably between 1 and 10, more preferably 1 and 8 micro drillings or between 4 and 5 micro drillings. The number of the micro blind holes per mm² surface is somewhat smaller because the drilling diameter is typically slightly larger compared with the micro drillings and is between 0.2 and 8, preferably between 0.2 and 6 or between 0.5 and 6 blind holes, preferably between 1 and 3 blind holes. Less or no continuous micro drillings or micro blind holes are located at the distal and proximal end of the catheter balloon. It is preferred when the continuous micro drillings or micro blind holes are as evenly as possible distributed over the central segment of the catheter balloon. The central segment refers to the part of the catheter balloon, which forms during dilatation a cylindrical shape. The micro drillings can be introduced into the balloon sleeve in checkerboard pattern or in the form of closed packing of spheres in space.

The catheter balloon is preferably expanded by means of a contrast medium solution, which is pressed into the balloon interior. This solution may also contain one or more anti-restenosis agents or anti-stenotic agents. By means of this solution a pressure in the interior of the catheter balloon is established, which first deploys the balloon, without stretching the balloon sleeve and at a certain pressure, which preferably corresponds to the nominal pressure, the balloon sleeve expands with fully unfolded balloon.

Therefore, the present invention also relates to a balloon catheter having a catheter balloon, wherein the catheter balloon has micro drillings and is covered with a metal mesh and the catheter balloon can be filled in its interior with an active agent solution, which can be applied through the balloon sleeve of the catheter balloon.

The present invention also relates to a balloon catheter having a catheter balloon, wherein the catheter balloon has micro drillings in form of continuous micro drillings or of micro blind holes and is covered with a metal mesh, and the catheter balloon can be filled in its interior with an active agent solution, which can be applied through the balloon sleeve of the catheter balloon.

The micro drillings are configured such that no or only very small quantities of liquid, particularly the contrast agent solution for balloon dilatation, can pass through the micro drillings. However, if the balloon sleeve of a “semi-compliant” or “compliant”—catheter balloon starts to stretch after full deployment and with further increase in pressure, thus, the micros drillings stretches, too, i.e. the micro drillings enlarge its inner diameter. Only from this pressure an appreciable amount of dilatation solution, i.e. particularly the contrast agent solution or the active agent solution with or without contrast medium can pass the micro drillings from the balloon interior to the exterior.

The balloon catheter according to the invention comprises a catheter balloon with micro drillings which so to speak, only opens at a defined pressure within the catheter balloon and releases a solution preferably an aqueous solution under pressure, which is preferably also used simultaneously for the dilatation of the catheter balloon and preferably contains further a contrast agent and at least one anti-restenosis agent or anti-stenose agent. Further, it is possible to apply a defined volume of this solution, and thus, based on the known concentration of the active agent in the solution to inject or rather to syringe under pressure a precise amount of the active agent into the vessel wall. This, for the first time, allows a study of the actually required lowest concentration of an anti-restenotic agent, because so far there is no possibility to apply a precisely defined amount of an anti-restenotic agent and in addition, to bring it directly to the site of action, i.e., to apply into the vessel wall. The active agent-containing balloon coatings of the prior art use, even if applied only locally, probably still an unnecessarily high dose of active compound.

The possibility to apply an anti-restenotic agent precisely metered at the site of action, where appropriate, sequentially in short intervals several times, allows for the first time a treatment of calcified stenosis and enables their dissolution.

Preferably, the micro drillings of a “semi-compliant” or “compliant”—catheter balloon open at a balloon internal pressure of 2 MPa, more preferably from 4 MPa, even more preferably of 5 MPa, even more preferably 6 MPa, even more preferably of 7 MPa, and most preferably of 8 MPa. In regard to a “non-compliant” catheter balloon the drillings deliver a significant dose of medicament only at a balloon internal pressure of 5 MPa, more preferably of 8 MPa, even more preferably of 10 MPa, and most preferably of 12 MPa. From this balloon internal pressure, the balloons are able to apply an aqueous solution and in particular an aqueous contrast agent solution containing at least one anti-restenosis agent or an anti-stenotic agent. Generally speaking, the catheter balloon according to the invention is suitable for application of an active agent solution, preferably an active agent-containing contrast agent solution or a solution of an active agent with a contrast agent at a balloon internal pressure of 2 to 15 MPa.

If an anti-restenotic agent and/or an anti-stenotic agent is added to the dilatation solution and preferably to the contrast agent solution, then the anti-restenotic agent and/or the anti-stenotic agent is injected by this way directly into the vessel wall and the surrounding tissue, because the release of the anti-restenotic agent and/or the anti-stenotic agent-containing solution occurs under high pressure. Thus, the anti-restenotic agent or anti-stenotic agent is applied directly to where he/it is needed. In addition, a great advantage is that the active agent is not located on the surface of the balloon, where it can be easily peeled off, but is applied as a solution from the interior of the balloon, which makes it possible to apply a defined amount of an active agent by a defined volume of an active agent solution. It is therefore preferred to use an active agent-containing contrast agent solution or an active agent solution containing a contrast agent, wherein said solution is also used for dilatation of the catheter balloon.

Iodated contrast agents common for angioplasty can be used as contrast agent solution for the inflation of the catheter balloons.

Anti-inflammatory, cytostatic, cytotoxic, anti-proliferative, anti-microtubule, anti-angiogenic, anti-neoplastic, anti-migrative, athrombogeneous and anti-thrombogenic agents are used as anti-restenosis agents.

Anti-inflammatory, cytostatic, cytotoxic, anti-proliferative, anti-microtubule, anti-angiogenic, anti-neoplastic, anti-migrative, athrombogeneous and anti-thrombogenic agents are preferably: vasodilator, sirolimus (rapamycin), somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastin, vincristin, vindesin, vinorelbin, etobosid, teniposid, nimustin, carmustin, lomustin, cyclophosphamide, 4-hydroxyoxycyclophosphamide, estramustin, melphalan, ifosfamide, tropfosfamide, chlorambucil, bendamustin, dacarbazin, busulfan, procarbazin, treosulfan, tremozolomide, thiotepa, daunorubicin, doxorubicin, aclarubicin, epirubicin, mitoxantrone, idarubicin, bleomycin, mitomycin, dactinomycin, methotrexate, fludarabin, fludarabin-5′-dihydrogen phosphate, cladribin, mercaptopurine, thiuoguanine, cytarabin, fluorouracil, gemcitabin, capecitabin, docetaxel, carboplatin, cisplatin, oxaliplatin, amsacrina, irinotecan, topotecan, hydroxy carbamide, miltefosin, pentostatine, aldesleukine, tretinoine, asparaginase, pegasparase, anastrozol, exemestane, letrozol, formestan, aminoglutethemide, adriamycin, azithromycin, spiramycin, cepharantin, 8-β-ergoline, dimethyl ergoline, agroclavin, 1-allylisuride, 1-allylterguride, bromerguride, bromocriptine (ergotaman-3′,6′,18-trione, 2-bromo-12′-hydroxy-2′-(1-methylethyl)-5′-(2-methylpropyl)-, (5′alpha)-), elymoclavin, ergocristine (ergotaman-3′,6′,18-trione, 12′-hydroxy-2′-(1-methylethyl)-5′-(phenylmethyl)-, (5′-alpha)-), ergocristinine, ergocornin (ergotaman-3′,6′,18-trione, 12′-hydroxy-2′,5′-bis(1-methylethyl)-, (5′-alpha)-), ergocorninine, ergocryptine (ergotaman-3′,6′,18-trione, 12′-hydroxy-2′-(1-methylethyl)-5′-(2-methylpropyl)-, (5′alpha)-(9Cl)), ergocryptinine, ergometrine, ergonovine (ergobasine, INN: ergometrine, (8beta(S))-9,10-didehydro-N-(2-hydroxy-1-methylethyl)-6-methyl-ergoline-8-carboxamide), ergosine, ergosinine, ergotmetrinine, ergotamine (ergotaman-3′,6′,18-trione, 12′-hydroxy-2′-methyl-5′-(phenylmethyl)-, (5′-alpha)-(9Cl)), ergotaminine, ergovaline (ergotaman-3′,6′,18-trione, 12′-hydroxy-2′-methyl-5′-(1-methylethyl)-, (5′alpha)-), lergotril, lisuride (CAS-Nr.: 18016-80-3,3-(9,10-didehydro-6-methylergolin-8alpha-yl)-1,1-diethylharnstoff), lysergol, lysergic acid (D-lysergic acid), lysergic acid amide (LSA, D-lysergic acid amide), lysergic acid diethylamide (LSD, D-lysergic acid diethylamide, INN: lysergamide, (83)-9,10-didehydro-N,N-diethyl-6-methyl-ergoline-8-carboxamide), iso lysergic acid (D-iso lysergic acid), lysergic acid amide (D-lysergic acid amide), isolysergic acid diethylamide (D-iso lysergic acid diethylamide), mesulergine, metergoline, methergine (INN: methylergometrine, (8beta(S))-9,10-didehydro-N-(1-(hydroxymethyl)propyl)-6-methyl-ergoline-8-carboxamide), methylergometrine, methysergide (INN: methysergide, (8beta)-9,10-didehydro-N-(1-(hydroxymethyl)propyl)-1,6-dimethyl-ergoline-8-carboxamide), pergolide ((8β)-8-((methylthio)methyl)-6-propyl-ergoline), proterguride and terguride, celecoxip, thalidomide, Fasudil®, cyclosporine, SMC-proliferation-inhibitor-2w, epothilone A and B, mitoxanthrone, azathioprin, mycophenolatmofetil, c-myc-antisense, b-myc-antisense, betulinic acid, camptothecine, PI-88 (sulphated oligosaccharide), melanocyte-stimulating hormone (α-MSH), activating protein C, IL1-β-inhibitor, thymosin α-1, fumaric acid and its ester, calcipotriol, tacalcitol, lapachol, β-lapachone, podophyllotoxin, betulin, podophyllic acid-2-ethylhydrazide, molgramostim (rhuGM-CSF), peginterferone α-2b, lanograstim (r-HuG-CSF), filgrastim, macrogol, dacarbazin, basiliximab, daclizumab, selectine (cytokine antagonist), CETP-inhibitor, cadherine, cytokine inhibitors, COX-2-inhibitor, NFkB, angiopeptine, ciprofloxacin, camptothecin, fluroblastin, monoconal antibodies, which inhibits muscle cell proliferation, bFGF-antagonist, probucol, prostaglandine, 1,11-dimethoxycanthin-6-on, 1-hydroxy-11-methoxycanthin-6-one, scopolectine, colchicine, NO-donors such as pentaerythrityltetranitrate and syndnoeimine, S-nitroso derivatives, tamoxifen, staurosporin, β-estradiol, α-estradiol, estriol, estrone, ethinyl estradiol, fosfestrol, medroxyprogesterone, estradiol cypionate, estradiol benzoate, tranilast, kamebakaurin and other terpenoide, which are used in cancer therapy, verapamil, tyrosine-kinase-inhibitors (tyrphostine), cyclosporine A and B, paclitaxel and ist derivatives such as 6-α-hydroxy-paclitaxel, baccatin, taxotere, synthetically produced as well as macrocyclische oligomers of carbon suboxide (MCS) obtained from natural sources and ist derivatives, mofebutazone, acemetacin, diclofenac, lonazolac, dapsone, o-carbamoyl phenoxyacetic acid, lidocaine, ketoprofen, mefenaminic acid, piroxicam, meloxicam, chloroquine phosphate, penicillamine, tumstatin, avastin, D-24851, SC-58125, hydroxy chloroquine, auranofin, sodium aurothiomalate, oxaceprol, celecoxib, β-sitosterine, ademetionine, myrtecain, polidocanol, nonivamide, levomenthol, benzocaine, aescin, ellipticin, D-24851 (Calbiochem), colcemide, cytochalasin A-E, indanocine, nocadazole, S 100 proteine, bacitracin, vitronectin-receptor antagonists, azelastin, guanidylcyclase-stimulator, tissue inhibitor of metallo proteinase-1 and 2, free nucleic acids, nucleic acids incorporated in virus capsules, DNA- and RNA-fragments, plasminogen-activator inhibitor-1, plasminogen-activator inhibitor-2, antisense oligonucleotides, VEGF-inhibitors, IGF-1, active agents from the group of antibiotics such as cefadroxil, cefazolin, cefaclor, cefotixin, tobramycin, gentamycin, penicillins such as dicloxacillin, oxacillin, sulfonamide, metronidazol, antithrombotics such as argatroban, aspirin, abciximab, synthetic antithrombin, bivalirudin, coumadine, enoxoparin, desulfated and N-reacetylated heparin, tissue plasminogen-activator, GpIIb/IIIa-plateled membrane receptor, factor Xₐ-inhibitor antibodies, interleukin inhibitors, heparin, hirudin, r-hirudin, PPACK, protamine, sodium salt of 2-methylthiazolidin-2,4-dicarboxylic acid, prourokinase, streptokinase, warfarin, urokinase, vasodilatators such as dipyramidol, trapidil, nitroprusside, PDGF-antagonists such as triazolopyrimidine and seramin, ACE-inhibitors such as captopril, cilazapril, lisinopril, enalapril, losartan, thioprotease inhibitors, prostacycline, vasiprost, interferone a, B and y, histamine antagonists, serotonin blocker, apoptose inhibitors, Apoptose regulators such as p65, NF-kB or Bcl-xL-antisense-oligo nucleotids, halofuginone, nifedipine, tocopherol, vitamin B1, B2, B6 and B12, folic acid, tranilast, molsidomin, teepolyphenole, epicatechin gallate, epigallocatechin gallate, boswellic acids and their derivatives, leflunomide, anakinra, etanercept, sulfasalazine, etoposide, dicloxacillin, tetracycline, triamcinolone, mutamycin, procainimide, D24851, SC-58125, retinolic acid, quinidine, disopyramide, flecainide, propafenone, sotolol, amidorone, natural and synthetically obtained steroide such as bryophylline A, inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenyl butazone and other antiviral agents such as acyclovir, ganciclovir and zidovudin, antimycotics such as clotrimazole, flucytosine, griseofulvin, ketoconazole, miconazole, nystatine, terbinafine, antiprozoal agents such as chloroquine, mefloquine, quinine, natural terpenoids such as hippocaesculin, barringtogenol-C21-angelate, 14-dehydro agrostistachin, agroskerine, agroskerine, agrostistachine, 17-hydroxy agrostistachine, ovatodiolide, 4,7-oxycycloanisomelic acid, baccharinoide B1, B2, B3 and B7, tubeimoside, bruceanole A, B and C, bruceantinoside C, yadanzioside N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, furthermore cymarin, apocymarin, aristolochic acid, anopterin, hydroxyanopterin, anemonin, protoanemonin, berberin, cheliburin chloride, cictoxin, sinococulin, bombrestatin A and B, cudraisoflavon A, curcumin, dihydronitidine, nitidine chloride, 12-beta-hydroxypregnadiene 3,20-dione, bilobol, ginkgol, ginkgolic acid, helenalin, indicin, indicin-N-oxide, lasiocarpin, inotodiol, glykoside 1a, podophyllotoxin, justicidin A and B, larreatin, malloterin, mallotochromanol, isobutyrylmallotochromanol, maquiroside A, marchantin A, maytansin, lycoridicin, margetin, pancratistatin, liriodenin, oxoushinsunin, aristolactam-all, bisparthenolidin, periplocoside A, ghalakinoside, urolic acid, deoxypsorospermine, psycorubin, ricin A, sanguinarin, acid of manwu wead, methylsorbifolin, sphatheliachromen, stizophyllin, mansonin, strebloside, akagerin, dihydrousambaraensin, hydroxyusambarin, strychnopentamine, strychnophylline, usambarin, usambarensin, berberin, liriodenin, oxoushinsunin, daphnoretin, lariciresinol, methoxylariciresinol, syringaresinol, umbelliferone, afromosone, acetylvismione B, desacetylvismione A, vismione A and B and sulphurous amino acids such as cystine as well as salts, hydrates, solvates, enantiomers, racemats, mixtures of enantiomers, mixtures of diastereomers; metabolits, prodrugs and mixtures of the aforementioned active agents.

Particularly suitable as anti-restenotic agents are paclitaxel and paclitaxel-derivatives such as e.g. taxotere, baccatine, 7-xylosyl-10-deacetyltaxol, cephalomannin, 10-deacetyl-7-epitaxol, 7-epitaxol, 10-deacetylcephalomannin, 7-desoxy-docetaxol, 7,8-cyclopropataxane, N-substituted 2-azetidones, 6,7-epoxy-paclitaxels, 6,7-modified paclitaxels, 10-deacetoxytaxol, 10-deacetyltaxol (of 10-deacetylbaccatin III), phosphonooxy- and carbonate derivatives of Taxol, Taxol 2′,7-dinatrium-1,2-benzoldicarboxylate, 10-deacetoxy-11,12-dihydrotaxol-10,12(18)-diene derivatives, 10-deacetoxytaxol, protaxol (2′- and/or 7-O-ester derivatives), 2′- and/or 7-O-carbonate derivatives of paclitaxel, fluortaxole, 9-deoxotaxane, 13-acetyl-9-deoxobaccatin III, 9-deoxotaxol, 7-deoxy-9-deoxotaxol, 10-deacetoxy-7-deoxy-9-deoxotaxol, sulphonated 2′-acryloyltaxol, sulphonated 2′-O-acylic acid taxol derivatives, succinyltaxol, 2′-gamma-aminobutyryl taxol, 2′-acetyltaxol, 7-acetyltaxol, 7-glycin-carbamate-taxol, 2′-OH-7-PEG(5000)-carbamate-taxol, 2′-benzoyl derivatives of paclitaxel, 2′,7-dibenzoyl-taxol, 2′-acetyltaxol; 2′,7-diacetyltaxol; 2′-succinyltaxol; 2′-(beta-alanyl)-taxol, ethylenglycol derivatives of 2′-succinyltaxol, 2′-glutaryltaxol, 2′-(N,N-dimethylglycyl)-taxol, 2′-(2-(N,N-dimethylamino)propionyl)taxol, 2′-orthocarboxybenzoyl-taxol, 2′aliphatic carboxylic acid derivatives of taxol, 2′-(N,N-diethylaminopropionyl)taxol, 7-(N,N-dimethylglycyl)taxol, 2′,7-di-(N,N-dimethylglycyl)taxol, 7-(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol, (N-debenzoyl-N-tert-(butoxycarbonyl)-10-deacetyltaxol, baccatin III, 10-deacetylbaccatin III, brevifoliol, yunantaxusin, taxusin, 14-beta-hydroxy-10-deacetybaccatin III, debenzoyl-2-acylpaclitaxel derivatives, benzoate paclitaxel derivatives, 18-side chain-substituted paclitaxel derivatives, chlorinated paclitaxel analoga, C4 methoxyether Paclitaxel derivatives, sulfonamide taxane derivatives, brominated paclitaxel analoga, Girard-taxol derivatives, nitrophenyl paclitaxel, 10-deacetylat substituted paclitaxel derivatives, 14-beta-hydroxy-10-deacetylbaccatin III derivatives, C7 taxane derivatives, 2-debenzoyl-2-acyltaxane derivatives, 2-debenzoyl paclitaxel derivatives, 2-acyl paclitaxel derivatives, 10-deacetyltaxol A, 10-deacetyltaxol B, 2-aroyl-4-acyl paclitaxel analoga, ortho-ester paclitaxel analoga, baccatin VII; baccatin VI; baccatin IV; 7-epi-baccatin III; baccatin V; baccatin I; baccatin A; epitaxol.

Furthermore, in particular rapamycin (Sirolimus) and rapamycin-derivatives are preferred, such as methylrapamycin, biolimus A9, deforolimus, everolimus, myolimus, novolimus, pimecrolimus, ridaforolimus, tacrolimus, temsirolimus, zotarolimus, 40-O-(2-hydroxyethyl)rapamycin (everolimus), 40-O-benzyl-rapamycin, 40-O-(4′-hydroxymethyl)benzyl-rapamycin, 40-O-[4′-(1,2-dihydroxyethyl)]benzyl-rapamycin, 40-O-allyl-rapamycin, 40-O-[3′-(2,2-dimethyl-1,3-dioxolan-4(S)-yl)-prop-2′-en-1′-yl]-rapamycin, (2′:E,4'S)-40-O-(4′,5′-dihydroxypent-2′-en-1′-yl)-rapamycin, 40-O-(2-hydroxy)ethoxycarbonylmethyl-rapamycin, 40-O-(3-hydroxy)propyl-rapamycin, 40-O-(6-hydroxy)hexyl-rapamycin, 40-O-[2-(2-hydroxy)ethoxy]ethyl-rapamycin, 40-O-[(3S)-2,2-dimethyldioxolan-3-yl]methyl-rapamycin, 40-O-[(2S)-2,3-dihydroxyprop-1-yl]-rapamycin, 40-O-(2-acetoxy)ethyl rapamycin, 40-O-(2-nicotinoyloxy)ethyl-rapamycin, 40-O-[2-(N-morpholino)acetoxy]ethyl-rapamycin, 40-O-(2-N-imidazolylacetoxy)ethyl-rapamycin, 40-O-[2-(N-methyl-N′-piperazinyl)acetoxy]ethyl-rapamycin, 39-O-desmethyl-39,40-O,O-ethylene-rapamycin, (26R)-26-dihydro-40-O-(2-hydroxy)ethyl-rapamycin, 28-O-methyl-rapamycin, 40-O-(2-aminoethyl)-rapamycin, 40-O-(2-acetaminoethyl)-rapamycin, 40-O-(2-nicotinamidoethyl)-rapamycin, 40-O-(2-(N-methyl-imidazo-2′-ylcarbethoxamido)ethyl)-rapamycin, 40-O-(2-ethoxycarbonylaminoethyl)-rapamycin, 40-O-(2-tolylsulfonamidoethyl)-rapamycin, 40-0-[2-(4′,5′-dicarboethoxy-1′,2′,3′-triazol-1′-yl)-ethyl]-rapamycin, 42-epi-(tetrazolyl) rapamycin (tacrolimus), and 42-[3-hydroxy-2-(hydroxymethyl)-2-methylpropanoate]rapamycin (temsirolimus).

Instead of or in addition to the anti-restenotic agents also active agents suitable for dissolving the calcifications can be released through the micro drillings in the balloon sleeve of the catheter balloon, which herein are referred to as anti-stenotic agent. Examples of anti-stenotic agents are decalcification agents and stenosis degrading agent.

This is the first time that not the result of a stenosis by dilatation of the narrowed vessel but the cause, that is, the calcified stenosis itself, is treated.

Hard calcifications consist of calcium carbonate. For dissolution of calcium carbonate, organic acids such as lactic acid, gluconic acid, propionic acid, acetic acid, citric acid, etc can be used as anti-stenotic agent, always in low biocompatible concentration, which form with calcium carbonate, water-soluble lactates, gluconates, propionates, acetates or citrates, or also inorganic acids. Because of the catheter balloons provided with continuous micro drillings or micro blind holes the anti-stenotic agents will be applied selectively. In a single angioplasty procedure the anti-stenotic agent can repeatedly be administered precisely dosed in short intervals several times in succession without the necessity that the balloon catheter must be removed from the vessel in between. The delivery in short time intervals is required to deflate the balloon catheter in between and to restore the blood flow in the vessel and, thus, avoid an undersupply of the heart. These multiple release of the active agent is also required in order to achieve a sufficient effect at the low concentrations of the anti-stenotic agent or due to the dilution by the blood.

With the frequently occurring diffuse coronary sclerosis, in which there are multiple coronary stenoses distributed over the coronary arteries, it is also possible to sequentially treat all calcified stenosis with a single active agent-eluting balloon catheter and at least nearly dissolve the slight calcifications. For this purpose the micro-drilled balloon catheter is placed at the calcified vascular sites and expanded with pressure for application of the active agent as described herein.

Furthermore, the surface of the catheter balloon is covered with a metal mesh. This metal mesh ensures the uniform dilatation of the catheter balloon and thus a uniform dilatation of the occluded vessel segment and minimizes the risk to overstretch the vessel.

In the prior art it is assumed erroneously that with insertion of a stent in the stenotic vessel segment, the stent has the function to mechanically maintain the vessel in open configuration after dilation and removal of the catheter balloon and to counteract a recoil, i.e. the counter-pressure, and the restoring force of the vessel. Herein, this view is referred to as not applicable. Due to the aforementioned view stents are also known as vascular support, which is also incorrect.

The stent provides rather a uniform dilatation and causes only incidentally a mechanical opening of the vessel segment. Therefore, it was also erroneously assumed with the introduction of the stent, that the stent keeps the vessel open and, thus prevents restenosis. In reality, however, the stent prevents the unilateral overstretching of the vessel during the balloon dilatation as set forth below.

The first time you inflate the balloon, the stent aligns itself immediately to the (calcified) vessel wall. At the start of the vessel dilation, the stent can certainly open unilaterally on the side not calcified. After complete dilation of the stent struts on the one side, the struts on the opposite side must stretch. Thus, the vessel is stretched uniformly and gently. Thereby the balloon cannot overstretch the vessel unilaterally.

The strength of the stent or of the stent struts is thus only important to avoid breaks of the struts during dilatation using a high balloon pressure. The high strength is not required to support the open vessel and to prevent recoil, i.e. to prevent re-narrowing of the vessel due to restoring forces. However, a supportive function and its positive effects cannot be completely ruled out. However, for this purpose significantly lower forces are sufficient, in particular because no external forces have an effect on the vessels, which would cause a re-narrowing and within the vessels the blood pressure counteracts re-occlusion of the vessel.

Because a stent is currently still not made of non-bioresorbable materials, and especially of metals and metal alloys, an implanted stent provides a permanent foreign compound in the patient's body, which can cause problems even after a long time (a few years), as the publications concerning the late thrombosis have shown.

Thus, one advantage of the present invention is that for the best possible reduction or prevention of restenosis, the implantation of a stent is not necessarily required.

Thus, the uniform dilatation of the catheter balloon is of particular importance. However, this can equally well be carried out with a metal mesh similar to a stent-like metal network, which is fixedly connected to the catheter balloon and cannot be displaced from the catheter balloon such as a stent.

Therefore, in order to achieve a uniform dilatation and thus, a disruption of calcified stenosis, without in parallel overstretching a non-calcified vessel segment and at the same time to limit the expansion of the “semi-compliant” or “compliant” catheter balloon, the catheter balloon according to the invention is provided with a metal mesh, which is fixedly mounted on the catheter balloon and is, after dilation, removed again from the patient together with catheter balloon.

According to the invention the design of the metal mesh is such that, in the deflated state of the balloon catheter, it fits as tightly as possible to the balloon surface, so that the diameter of the catheter balloon in the deflated state (also referred to as a compressed or folded state) is as small as possible. Nevertheless, the metal mesh has to be designed such that it allows the dilatation of the catheter balloon, i.e. that it can be expand such as a stent, without occurrence of strut breaks. The dilatation of the metal mesh is not carried out by elongation of the struts of the metal mesh, but by a dilatation of the mesh structure of the metal mesh as well as for a stent.

Concerning a “semi-compliant” catheter balloon the metal mesh is preferably not maximally dilated at nominal pressure, but allows at this pressure even a slight further expansion so that the fully deployed catheter balloon can stretch, with further increase in pressure, to a certain extent even beyond the nominal pressure.

Regarding a “non-compliant” PTCA catheter balloon the balloon catheter as well as the metal mesh are preferably dilated at the maximum at nominal pressure.

Even concerning a “compliant” PTCA catheter balloon, at nominal pressure the metal mesh is preferably stretched to a maximum or approximately to a maximum.

However, the struts of the metal grid must be meshed particularly closely. The distance between two metal struts should be less than 2.0 mm in the fully expanded state, preferably less than 1.5 mm, and more suitably less than 1.0 mm, so that the balloon cannot protrude between the struts, as for example, it is the case with the product Chocolate® of the company TriReme Medical Inc.

This means that the metal mesh located around the catheter balloon expands, too, during the inflation of the catheter balloon. During this expansion, the metal mesh fits still as close as possible to the surface of the catheter balloon. This results in achievement of a uniform expansion of the catheter balloon and a unilateral overstretching of the vessel, in particular a blood vessel, remains.

Moreover, this uniform dilatation of the catheter balloon also results in the desired break-up of the stenosis, and in particular a calcified or heavily calcified stenosis.

With the expansion of the metal mesh during dilatation, the metal struts of the metal mesh shift relative to each other, thereby creating shear forces as well as they are formed during the expansion of a stent. These shear forces also support the break-up of calcified stenosis, thus reduce the overstretching of the non calcified vessel segment, avoid dissection and thereby at best reduce the restenosis rate.

In addition, regarding a “semi-compliant” balloon catheter, the metal mesh according to the invention has yet another function. At the nominal pressure, the metal mesh is not expanded to its maximum and a slight further expansion to a maximum diameter of the metal mesh is possible.

The catheter balloon can therefore be fully deployed and then has the possibility to stretch upon further increase in pressure, i.e., there is a further enlargement of the diameter, which is caused by the stretching of the sleeve of the catheter balloon and no longer by the unfolding of the folded catheter balloon.

During this further expansion of the “semi-compliant” catheter balloon, which arises due to elongation of the material of the catheter balloon, also the micro drillings stretch so that they increase their internal diameter and a solution contained inside the balloon, which contains preferably at least one anti-restenosis agent and/or an anti-stenotic-agent, is injected under pressure into the vessel wall and the surrounding tissue. In the same way also the opening of the micro drillings in a “compliant” PTCA catheter balloon can take place.

In order to limit this further enlargement of the balloon catheter due to elongation of the “semi-compliant” and in particular of the “compliant” balloon material the metal mesh can be expanded only up to a defined diameter, which then prevents the further increase of the catheter balloon.

Thus, the metal mesh of the present invention limits the further expansion of the catheter balloon and thus defines a maximum diameter for the maximal expanded catheter balloon. Concerning a “semi-compliant” as well as a “compliant” catheter balloon this maximum diameter predetermined by the metal mesh is in the range in which the “semi-compliant” catheter balloon can stretch after exceeding the nominal pressure. Preferably, this maximum diameter is the diameter that a “semi-compliant” balloon catheter has at a pressure that is above, and preferably 0.5 MPa higher than, the nominal pressure and lower than 20 MPa, preferably lower than 18 MPa. At such a maximum diameter, the balloon catheter is fully deployed so that the stenosis and especially the calcified stenosis uniformly braked and in addition, the balloon sleeve is slightly stretched, resulting in the opening of the micro drillings and the delivery of the solution such as an active agent-containing physiological solution or an active agent-containing contrast agent solution.

Further, the metal mesh is configured such that the metal mesh can be expanded from an initial first inner diameter to a maximum second inner diameter, with the dilatation of the catheter balloon and during the deflation of the catheter balloon can preferably be reduced to the first inner diameter, or may be reduced to an inner diameter, which is as similar as possible to the first inner diameter.

In addition, it is preferred that the metal mesh is not so loosely meshed as with the product Chocolate® by the Company TriReme Medical Inc., so that in the individual meshes of the metal mesh, pillows can be formed because the sleeve of the catheter balloon is squeezed out within the mesh of the metal mesh and the struts of the metal mesh tie up the catheter balloon, whereby valleys are formed in the balloon sleeve along the struts of the metal mesh. In the aforesaid product of the prior art, the contact surface of the balloon surface with the vessel wall should be maximized, so that the balloon sleeve should ooze out of the meshes of the metal mesh, so that as much as possible of the active agent can be transferred from the balloon surface to the vessel wall.

Since it is not envisaged but also not excluded that the catheter balloon according to the invention is provided with an active agent containing coating, a largest possible contact area between the balloon surface and the vessel wall is not important for the present invention, because the active agent is applied from the interior of the catheter balloon through the micro drillings (either continuous micro drillings or micro blind holes opened by rupturing of the membrane).

According to the invention, the metal mesh is closely meshed and more comparable to the metal mesh of a coronary stent. According to the invention, it is preferred that the balloon sleeve cannot protrude through the meshes of the metal mesh.

The metal mesh is made of an elastic material, preferably made of the super elastic binary material nickel-titanium (NiTi). The metal mesh can be braided or woven using thin NiTi wires. But preferably, it is laser cut from a NiTi tube, since using laser cut the grid style or design contours can flexibly fitted to the requirements along the length of the catheter balloon such as a very tight fit on the balloon at the proximal and distal end, limiting the maximum of the expanded diameter, etc. In order to smooth the surface, the metal mesh is electro-polished but only slightly so that the strut edges remain sharp on the outside (abluminal side of the grid) and facilitate the break-up of calcified stenosis.

Since the metal mesh should remain on the catheter balloon and be pulled out with the removal of the catheter balloon, it must be firmly anchored to the catheter balloon on the one hand and also on the other hand, it should be contracted as far as possible during the deflation of the catheter balloon, so that it can again be easily removed together with the catheter balloon.

Therefore, it is preferred that the metal mesh at the distal or proximal end, and further preferably at the distal and proximal end is fix connected to the catheter balloon, for example, as there the network firmly encloses the balloon ends.

A contraction of the metal mesh during the deflation of the catheter balloon may be accomplished by using a metal or a metal alloy having superelastic properties for the metal mesh. A metal or a metal alloy having super-elastic properties is a shape memory alloy, wherein said metal or said metal alloy preferably assumes a certain predetermined shape at a certain temperature or a certain temperature range.

A metal alloy with super elastic properties suitable for the metal mesh is, for example, a binary nickel-titanium alloy. Particularly preferred is a nickel-titanium alloy with a weight fraction of about 55% nickel, which comprises super elastic properties at a transformation temperature of typically 10° C. to 20° C. below the body temperature (37° C.). It is crucial that shape memory alloys, such as NiTi, possess superelastic characteristics. According to the invention, therefore, all the super-elastic metal alloys and preferably all superelastic metal alloys with shape memory can be used.

If the metal mesh for the catheter balloon is prepared from such a metal or such a metal alloy with shape memory so, that the metal mesh is, for example at 20° C., in the form that it fits as close as possible to the compressed or deflated catheter balloon, then under mechanical deformation and at a temperature of or in the region of 37° C., it tries to elastically return in said form that it has been given at a temperature of 20° C.

This means that the metal mesh was given a form for a temperature of 20° C. which fits as closely as possible to the folded or compressed catheter balloon, making it possible that the catheter balloon can be guided or pushed through the vessels or blood stream as good as possible. If then, the catheter balloon is dilated by establishing a corresponding pressure in the catheter balloon, the metal mesh yields to this pressure and under this force it can be mechanically deformed elastically until a maximum expansion of the metal mesh has been reached, which then prevents the additional expansion of the catheter balloon after complete deployment during further pressure increase.

If the catheter balloon is then fully deployed and has been dilated under the rising pressure to open the continuous micro drillings or to open the micro blind holes by rupturing of the membrane being located at the bottom and to release an active agent solution through the opened micro drillings, the deflation of the catheter balloon is carried out by creating a negative pressure in the interior of the catheter balloon. But the deflation of the catheter balloon does not necessarily results in a contraction of the metal mesh. The metal mesh contracts again at the temperature of 37° C. due to the super-elastic properties. This means, because there is a temperature of about 37° C. in the human body, the metal mesh seeks to form into the original shape being tight-fitting to the compressed catheter balloon. As long as a corresponding pressure in the interior of the catheter balloon has forced a mechanical expansion of the metal mesh, the restoring forces of the metal mesh could not overcome the internal pressure in the catheter balloon and the metal mesh was and remained dilated. In the case of deflation of the catheter balloon, this positive pressure does no longer exists in the interior of the catheter balloon, and the restoring forces of the metal net acting at 37° C. or near 37° C. come into play, and cause a contraction of the metal mesh close to the original shape. Thus, the catheter balloon with the metal mesh reduces its diameter such that it can be withdrawn from the vascular system. Diameter refers to the maximum diameter, which is produced by the catheter balloon and the metal mesh, which corresponds in general with the distance between two opposed struts of the metal mesh with maximum distance from each other.

Furthermore, the present invention relates to balloon catheter having a catheter balloon, wherein the catheter balloon is covered with a metal mesh and has micro drillings, which are obtainable by treatment of a catheter balloon without micro drillings by means of laser in order to generate the micro drillings and subsequent mounting of the metal mesh on the catheter balloon.

A further aspect of the present invention is directed to the production of balloon catheter having a catheter balloon, wherein the catheter balloon has micro drillings and is covered with a metal mesh and the catheter balloon is configured to receive an active agent solution which can be applied through the micro drillings comprising the steps of:
A) Providing a balloon catheter having a catheter balloon without channels, drillings or openings in the balloon sleeve of the catheter balloon,
B) introducing micro drillings using a laser into the balloon sleeve of the catheter balloon and
C) attaching a metal mesh to the catheter balloon.

For laser drilling of catheter balloons, the balloon is inflated, i.e. unfolded, with an inert gas. The deployed balloon is held in a device, to accurately position it under the focused laser beam. In particular, the focus position, that is, the position of the smallest focal point is crucial for achieving such small micro drillings. The wafer-thin balloon sleeve is perforated in the percussion method, i.e. successively pierced by several ultra short laser pulses. The pulse energies required are very low, with 0.05 to 10 μJ depending on the balloon material. The pulse frequencies are in the kHz range, with which the drillings can be incorporated in a fraction of a second. The laser wavelength is selected depending on the balloon material from 193 nm to about 2000 nm.

The inventive micro drilled balloon catheter with metal mesh offers significant advantages over the balloon catheters of the prior art:
1. The metal mesh with super elastic properties stretches together with the catheter balloon and ensures a uniform expansion of the vessel and in particular of calcified vessel segments. With the release of the pressure in the interior of the catheter balloon the metal mesh with shape memory contracts again, too, and is removed again from the vessel together with the balloon catheter.
2. Also strongly calcified vessel segments are uniformly and homogeneously dilated so that the calcified stenosis break and over extension of a non-calcified vessel segment is avoided.
3. If a support of the vessel wall is required, for example, because of a dissection, subsequently, a balloon expandable stainless steel or CoCr stent or preferably a bioabsorbable polymer or metal stent can be implanted.
4. In principle, however, the use of a stent can be avoided, if the stent should have primarily the function of the uniform dilatation of the stenosis and the permanent implantation of a foreign body in the blood vessels of the patient should be avoided, whereby the risk of late thrombosis is excluded, too.
5. Only with a certain pressure, an appreciable amount of the active agent containing solution can be injected from the interior of the catheter balloon passing the micro drillings directly into the vessel wall, so that the anti-restenotic agent is not washed away by the blood stream, but is injected directly into the site of action.
6. In addition, it is now for the first time possible to apply by this way, a precisely defined amount of an active agent.
7. Since the active agent is not located on the surface of the catheter balloon, there are also no problems regarding the durability (shelf life) because the active agent is introduced into the balloon catheter only in the moment of the dilatation. The micro-drilled balloon catheters according to the invention having a metal mesh can therefore in principle be stored without limit.
8. Shortly before dilatation, a suitable active agent can be selected and that even locally by the attending cardiologist.
9. As the inventive micro-drilled balloon catheter with the metal mesh is free of active agent, there are lower approval hurdles, too.
10. No active agent is lost during advancement of the balloon catheter up to the stenosis.
11. Once inserted, the balloon catheter can be used at several stenotic vessel segments and over again, a defined amount of active agent can be delivered.
12. The active agent is literally injected into the vessel wall and does not have to diffuse into, which always leads to loss of active agent in the blood vessel.

Furthermore, other clinical applications arise for the micro-drilled balloon catheter with metal mesh according to the invention.

Particularly preferred is a combination of the application of the balloon catheter according to the invention with a subsequent implantation of a bioresorbable stent. A vessel stenosis dilated once with the inventive balloon catheters and treated with an active agent can subsequently be supplied with a, preferably bioresorbable stent, if a vessel support is transitionally necessary.

The bioresorbable stents made of polymer or magnesium generally have a low material strength and are therefore less suitable for the balloon dilatation for the following reasons:

The struts fit at the beginning of the expansion to the vessel wall and follow the uneven expansion of the vessel, which they cannot avoid due to their low strength. Due to the low material strength some struts may even break. After expansion of the bioabsorbable stent, one observes typically strut overlaps, in particular an uneven distribution of the struts in the periphery (malapposition) and partially strut fractures. A uniform circular vessel dilatation cannot be ensured.

But during “free” dilatation of bioresorbable stents (in air) a uniform break less dilatation can be seen.

Comparably, in a pre-stretched vessel (predilatation), the bioresorbable stent can deploy uniformly and freely without large forces and raise the required low supporting force evenly.

Because the drug has been administered via the micro-drilled catheter balloon, an active agent coating of the bioresorbable stent is not required, which greatly simplifies the product approval.

The bioresorbable materials have been a low strength and therefore a large strut thickness, with the result that these stents have a large diameter (“profile”) in the crimped state. The bioresorbable stents can therefore only be pushed in relatively large vessel openings or into stenosis with large lumen. There are no restrictions for vessels being pre-dilated with the balloon catheter according to the invention.

Therefore, the present invention also relates to a method for applying an anti-restenotic agent or an anti-stenotic agent by using a catheter balloon with micro drillings and a metal mesh, wherein said catheter balloon is inflated by means of a contrast medium solution containing the anti-restenotic agent or the anti-stenotic agent, and wherein with increasing internal pressure in the catheter balloon first the folded catheter balloon unfolds and after full deployment with further increase in pressure an elongation of the balloon sleeves takes place, which causes opening of the micro drillings and a release of the contrast agent solution containing the anti-restenotic agent or the anti-stenotic agent through the micro drillings, wherein the elongation of the balloon sleeve is limited by the metal mesh. This allows a defined amount of an anti-restenotic agent or an anti-stenotic agent to be applied at the site of the balloon dilatation and preferably being injected directly into the vessel wall.

The present invention refers also to a method for application of an active agent solution containing a contrast medium or an active agent containing contrast agent solution comprising the steps.
A) Providing a balloon catheter with a catheter balloon, wherein the catheter balloon has micro drillings and is covered with a metal mesh and the catheter balloon is formed for receiving of an active agent solution, which can be applied through the micro drillings,
B) dilatation of the catheter balloon using the active agent solution up to a pressure that opens the micro drillings to apply a predefined volume of the active agent solution and
C) maintaining the pressure until the predefined volume of the active agent has been applied.

### DESCRIPTION OF THE FIGURES

FIG. 1: shows a catheter balloon in a compressed or inflated state, which is laid in folds. The circle around the compressed or deflated catheter balloon denotes the idealized round shape of the catheter balloon for the purpose of determining the diameter.

FIG. 2: Fluid delivery of non-compliant balloons

FIG. 2 shows the flow rate of water in 30 seconds of a non-complaint balloon having 200, 400 and 600 drillings with a diameter of 2.0 μm±0.5 μm.

FIG. 3: Fluid delivery of semi-compliant balloons

FIG. 3 shows the flow rate of water in 30 seconds of a semi-complaint balloon having 200 and 600 drillings with a diameter of 2.0 μm±0.5 μm.

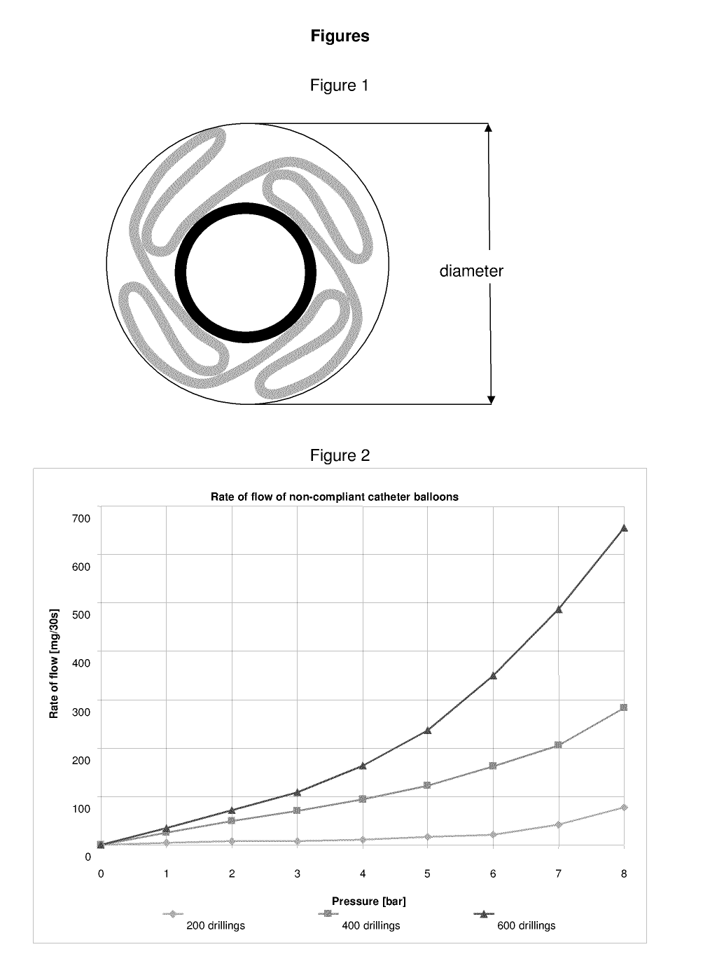

### EXAMPLES

### Example 1

### Manufacture of Catheter Balloons with Continuous Drillings and Fluid Delivery

The catheter balloons are inflated e.g. using nitrogen, and continuous drillings are made in the balloon sleeve using an ultra short laser (USP-laser). The micro drillings pass vertically trough the balloon sleeve in direction of the longitudinal axis of the catheter balloon. The micro drillings have a diameter of 2.0 μm±0.5 μm.

The fluid delivery of non-compliant and semi-compliant balloons was measured dependent on the number of drillings and the fluid pressure

### Example 2

### Manufacture of Catheter Balloons with Micro Blind Holes and Fluid Delivery

The catheter balloons are inflated e.g. using nitrogen and first of all the balloon wall thickness was measured with an accuracy of less than 1 micron. The balloon wall thickness is critical for setting the Laser abrading process or the remaining membrane wall thickness on the balloon inside.

The work is done by an ultra short laser (USP-laser). The laser beam diameter in the laser beam focus is adjusted on a diameter of 10 μm by optical elements. Each laser pulse gradually removes a small defined layer of the balloon wall thickness so that a cylindrical bore is formed with a diameter of 10 μm. The number of laser pulses determines the depth of the micro blind hole or the remaining wall thickness (membrane) at the hole bottom. Depending on the previously measured balloon wall thickness, the number of laser pulses is chosen such that a membrane thickness of 3-4 μm remains on the balloon inside.

At a fluid internal pressure of 4-5 bar (4-5 MPa), the membrane bursts, so that liquid delivery from the catheter balloon occurs.

### Example 3

### Determination of the Applied Volume of the Dilatation Solution

To determine the applied volume of a dilatation solution a non-complaint balloon with 200, 400 and 600 continuous drillings as well as a semi-complaint balloon with 200 and 600 continuous drillings were examined. The micro drillings had a diameter of 2.0 μm±0.5 μm s, respectively. In practice, the drug solution described herein or preferably the drug-containing contrast media solution is used as dilatation solution. In Example 3, for practical reasons, purified water (distilled water) was used.

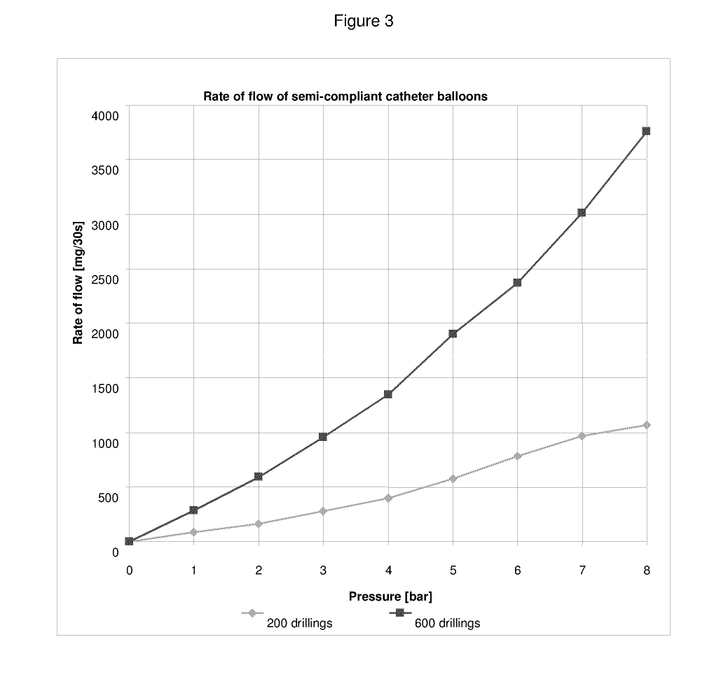

The used catheter balloons according to the invention were gradually dilated to 8 bar (8 MPa) with water as dilatation solution. In each case at 1 bar (1 MPa), 2 bar (2 MPa), 3 bar (3 MPa), 4 bar (4 MPa), 5 bar (5 MPa), 6 bar (6 MPa), 7 bar (7 MPa), and 8 bar (8 MPa) the volume of applied dilatation solution was determined over a period of 30 seconds. This experiment was repeated three times. The results are shown in FIGS. 2 and 3.

Regarding a non-complaint balloon with a total of 200 micro drillings per balloon, in FIG. 2, it can be seen that at 8 bar (8 MPa) 78 mg dilatation solution can be applied within a dilatation interval of 30 seconds. The continuous drillings have a diameter in the range of 1.5 μm to 2.5 μm. With 400 micro drillings per balloon there are 283 mg and with 600 micro drillings per balloon 656 mg (see FIG. 2). The corresponding values for a semi-complaint balloon are shown in FIG. 3.

In a comparison of FIGS. 2 and 3, the different scale of the Y-axis is to be observed (flow rate). The semi-complaint balloons release significantly more liquid than the non-complaint balloons, because the drillings dilate more under pressure. The semi-compliant balloons have to be provided with correspondingly less continuous micro drillings or suitable for higher fluid doses, e.g. active agent dosage for example for release of anti-stenotic agents to be administered with higher dosage.

### Example 4

### Use of a Catheter Balloon According to the Invention

A commercially available conventional PTCA balloon catheter is used. The catheter balloon is uncoated and is made of polyamide (trade name nylon) or PET. It is a “non-compliant”-catheter balloon, which is virtually inelastic. The increase in the diameter of this “non-compliant”-catheter balloon is based on the deployment of the catheter balloon wherein the stretching of the sleeve material of the catheter balloon plays no role. After complete deployment a minimal increase in the diameter of the catheter balloon can be achieved by further increasing the internal pressure in the catheter balloon.

The catheter balloon (ø 3.0×20 mm) is inflated, e.g. with nitrogen, and with USP laser continuous micro drillings are made in the balloon sleeve. The micro drillings passing vertically through the balloon sleeve along the longitudinal axis of the catheter balloon. The continuous micro drillings have a diameter of 1.7 μm±0.3 μm. 1.5 μm drillings are formed per mm² surface of the fully expanded catheter balloon.

The thus processed catheter balloon is then covered in a completely deflated condition with a tight-fitting metal mesh made of a nickel-titanium alloy (such as Nitinol®), which is tightly connected with the catheter balloon at the distal and proximal ends of the catheter balloon. Therefore a loop of the metal mesh is tightly wrapped around the distal end of the balloon and another loop is tightly wrapped around the proximal end of the balloon. In the area between the distal and the proximal end of the catheter balloon, the metal mesh is formed like a stent with reticulated struts, so that this area can expand during dilatation of the catheter balloon.

The metal mesh of, for example Nitinol®, increases the diameter of the fully deflated and folded catheter balloon only slightly. The completely deflated and folded catheter balloon without metal mesh has a diameter of 0.8 mm, where the fully deflated and folded catheter balloon with a metal mesh has a diameter of 1.0 mm.

The catheter balloon is, as usual, advances in the patient through the groin up to the heart region over a guide wire and placed in the stenotic and calcified vessel segment. Now, the dilatation of the catheter balloon is carried out by pumping an aqueous contrast agent solution of the contrast agent iopromide into the interior of the catheter balloon. The contrast agent solution is commercially available under the name Ultravist®. The active agent paclitaxel has been added to the contrast agent solution in a concentration of 0.5-2.0 μg per ml.

With increasing pressure in the interior of the catheter balloon, the catheter balloon begins to unfold, wherein the micro drillings are still closed such that no significant leakage of paclitaxel containing contrast agent solution can be observed. Upon further increase in pressure, the catheter balloon continues to enlarge its diameter uniformly cased by the surrounding Nitinol mesh that expands to the same extent than the catheter balloon. Thus, the calcified stenosis is effectively broken up and an overdilatation of the non calcified vessel area is effectively prevented. At a pressure of 6.0 MPa, the catheter balloon is fully deployed (nominal pressure). Now, the pressure in the interior of the catheter balloon is increased to 8 MPa. This results in that the paclitaxel containing contrast agent solution flows pressurized through the continuous micro drillings and is literally injected into the vessel wall. In addition, at this pressure, the metal mesh is maximally expanded and mechanically limits further dilatation of the sleeve material of the balloon if the pressure in the catheter balloon is further increased so that no further increase of the diameter of the catheter balloon can take place. Over a period of 20 seconds, a drug dose of approximately 2 to 10 μg of paclitaxel per millimeter vessel wall length (corresponding to 0.20 to 1 μg per mm² vessel wall surface) is injected in the vessel wall.

Thereafter, the pressure in the balloon is lowered to 0.5 MPa, the balloon catheter is easily moved and dilated again until a pressure of 8 MPa is reached. Over a period of additional 20 seconds, a drug dose of approximately 2 to 10 μg of paclitaxel per millimeter vessel wall length is applied.

After the second dilatation, the pressure in the catheter balloon is released again. With decreasing pressure, also the catheter balloon starts to decrease again and places in folds. The surrounding metal mesh also contracts due to the restoring forces and clings still to the surface of the catheter balloon. A substantial deflation of the catheter balloon is finally achieved by generating a negative pressure in the interior of the catheter balloon. The metal mesh was given the original shape fitting close to the fully folded and packed catheter balloon by heat treatment steps, therefore, the metal mesh aims to its original shape when eliminating the pressure inside the catheter balloon and fits again close to the deflated catheter balloon and can thus be easily removed from the patient together with the catheter balloon.

Likewise, if several stenotic vessel segments of the same patients are treated, this can even be carried out using the same catheter balloon and by targeted drug delivery through the micro drillings at various sites in the vascular system.

1. A balloon catheter with a catheter balloon, wherein the catheter balloon has micro drillings and is covered with a metal mesh, and the catheter balloon is formed for receiving an active agent solution, which can be administered through the micro drillings.

2. The balloon catheter according to claim 1, wherein the catheter balloon is a non-compliant or a semi-compliant or a compliant catheter balloon.

3. The balloon catheter according to claim 1, wherein the metal mesh is made of a superelastic metal or a superelastic metal alloy.

4. The balloon catheter according to claim 1, wherein the metal mesh is made of a nickel-titanium-alloy.

5. The balloon catheter according to claim 1, wherein the metal mesh is dilatable from an initial first inner diameter to a maximum second inner diameter with the dilatation of the catheter balloon and with deflation of the catheter balloon the size of the metal mesh is reduced to the first inner diameter again.

6. The balloon catheter according to claim 1, wherein after maximal dilatation the metal mesh prevents further dilatation of the catheter balloon with increasing internal pressure.

7. The balloon catheter according to claim 1, wherein the catheter balloon has between 0.2 and 10 micro drillings per mm² of its surface.

8. The balloon catheter according to claim 1, wherein the micro drillings are continuous drillings or micro blind holes.

9. The balloon catheter according to claim 8, wherein the continuous drillings have a diameter of 0.5 μm-5.0 μm and the micro blind holes have a diameter of 2.0 μm to 20.0 μm.

10. The balloon catheter according to claim 8, wherein the catheter balloon has between 0.2 and 6 micro blind holes or between 1 and 10 continuous drillings per mm² of its surface.

11. The balloon catheter according to claim 1, wherein the micro drillings in the catheter balloon are made using laser treatment.

12. The balloon catheter according to claim 1, wherein the micro drillings in the catheter balloon are suitable for administration of the active agent solution at a balloon internal pressure of 2 to 15 MPa.

13. The balloon catheter according to claim 12, wherein the active agent solution is a solution of an anti-stenotic agent or an anti-proliferative, anti-angiogenic or anti-restenotic agent.
